SEQUENCE LISTING (9 sequences)

SEQ ID NO: 1 - DNA, 21 bp, Artificial Sequence
  HLA-DR4 alpha forward primer
aatcctgaccaatcaggcgag

SEQ ID NO: 2 - DNA, 21 bp, Artificial Sequence
  HLA-DR4alpha Reverse Primer
gcggaagaggtgatcgtccct

SEQ ID NO: 3 - DNA, 20 bp, Artificial Sequence
  HLA-DR4 beta Forward
gtttcttggagcaggttaaa

SEQ ID NO: 4 - DNA, 20 bp, Artificial Sequence
  ctgcactgtg aagctctcac
ctgcactgtgaagctctcac

SEQ ID NO: 5 - DNA, 20 bp, Artificial Sequence
  HLA-A2 forward primer
tccatgaggtatttcttcac

SEQ ID NO: 6 - DNA, 19 bp, Artificial Sequence
  HLA-A2 reverse primer
ggcctcgctctggttgtag

SEQ ID NO: 7 - DNA, 20 bp, Artificial Sequence
  B2m-COMMON forward primer
tatcagtctcagtgggggtg

SEQ ID NO: 8 - DNA, 21 bp, Artificial Sequence
  B2m-Mutant reverse primer
tctggacgaagagcatcaggg

SEQ ID NO: 9 - DNA, 20 bp, Artificial Sequence
  B2m wildtype reverse primer
ctgagctctgttttcgtctg

(19) United States
(12) Patent Application Publication
(10) Pub. No.: US 2013/0291134 A1
(43) Pub. Date: Oct. 31, 2013

(54) HUMANIZED TRANSGENIC MOUSE MODEL

(71) Applicants: Sofia a. Casares, Potomac, MD (US); Tepdpr D. Brumeanu, Potomac, MD (US)

(72) Inventors: Sofia a. Casares, Potomac, MD (US); Tepdpr D. Brumeanu, Potomac, MD (US)

(21) Appl. No.: 13/592,000

(22) Filed: Aug. 22, 2012

Related U.S. Application Data
(63) Continuation-in-part of application No. PCT/US11/01656, filed on Sep. 26, 2011.
(60) Provisional application No. 61/386,118, filed on Sep. 24, 2010.

(51) Int. Cl. A01K 67/027; C07K 16/06; G01N 33/50; G01N 33/68

(52) U.S. Cl. CPC A01K 67/0278; G01N 33/6854; C07K 16/06; G01N 33/505
USPC 800/6; 800/9; 800/22; 435/7.92; 530/388.15; 435/29

(57) ABSTRACT

This invention relates to a transgenic animal model for testing immunogenicity and protective efficacy of human vaccines and the method for generating such a multi-transgenic animal. This invention also relates to methods for screening compositions for human vaccine development. More specifically, the present invention relates to a mouse model capable of expressing human leukocyte antigens DR4 and A2, and/or human costimulatory molecules (CD80) which upon infusion of human HLA-matched hematopoietic stem cells develop a functional human immune system able to respond to vaccination with human vaccines. The invention also relates to method of producing human antibodies specific for a desired antigen using the transgenic mouse.

### CROSS-REFERENCE TO RELATED APPLICATION

This application claims priority to U.S. Patent Application No. 61/386,118 filed 24 Sep. 2010 and related to PCT application PCT/US11/001,656, filed on 26 Sep. 2011.

### BACKGROUND

Human vaccine research often requires in vivo analysis to test the immunogenicity, protective efficacy, and toxicity of certain vaccine candidates. However, in vivo human study is severely limited due to ethical and technical constraints. There is a growing need for an animal model to carry out in vivo studies of human cells, tissues and organs, without putting individuals at risk. Many important studies have been carried out using mice as an animal model in research of complex human biological systems. The development of “humanized mouse”, provide an opportunity to study human biological processes in vivo that would otherwise not be possible. In recent years, many mouse models were developed with different genetic manipulations designed to meet the needs of in vivo studies of human systems (See Table 2). Although, current mouse models permit long-term hematopoiesis of human B cells (2, 3), they only allow poor development of functional human T cells (2-5). These mouse models also fail to develop serum levels of human IgM and IgG comparable to that of human blood (3). Furthermore, upon vaccination, infection, or transplantation, current humanized mouse models do not elicit immune responses in the same extent as vaccinated or infected humans (2-5, 7-9).

In previously applications (U.S. Patent Application No. 61/386,118 and PCT application PCT/US11/001,656) we disclosed a humanized mice capable of expressing HLA class II (DR4) molecules and knockout (KO) for Rag and IL2Rgc genes (DRAG). The DRAG mice when infused with HLA-matched human hematopoietic stem cells can develop a functional human immune system consisting of human CD4 T cells, CD8 T cells, B cells, and dendritic cells (PLoS ONE 6: e19826, 2011, PCT/US11/001,656).

However, the DRAG mice do not express human HLA class I molecules. As such, the human CD8 T cells developed by DRAG mice are likely to be restricted by mouse MHC class I molecules. Since CD8 T cells are important effector cells for elimination of pathogens, a fully humanized mouse, or a mouse-human chimera, as described in this application, can overcome the constraints of the DRAG and other current mouse models, and may become an important research tool for in vivo studies.

The new humanized mouse model (DRAGA) is capable of co-expressing HLA class I and class II molecules in such a way that the human CD8 T cells developed by these mice are only restricted by human MHC class I molecules. Three other mouse strains are also generated in the process of creating the DRAGA mouse, with each displaying additional phenotypes. DRAGAB and DRAGAB80 also have knockout for mouse beta-2-microglobulin (B2m). The B2mKO mutation prevents expression of mouse MHC class I molecules and ensures that all human CD8 T cells are restricted by HLA class I molecules. DRAG80 and DRAGAB80 also express human CD80 in the pancreatic beta cells (hCD80), which may facilitate the autoimmune attack of human T cell on pancreas and development of type 1 diabetes. All four new mouse strains of this application are summarized below.

TABLE 1
New Mouse Strains

| Name of Strain | Mutate allele |
|---|---|
| DRAGA | HLA-DR4, HLA-A2.1 |
| DRAGA80 | Human CD80, HLA-DR4, HLA-A2.1 |
| DRAGAB | HLA-DR4, HLA-A2.1, knockout for mouse B2m |
| DRAGAB80 | Human CD80, HLA-DR4, HLA-A2.1, Knockout for |
| | mouse B2m |

Efficacy of passive transfer of human hyper-immune sera containing polyclonal immunoglobulins (Igs) has been widely proven in clinical trials for infectious diseases, including malaria [15]. However, the use of human hyper-immune sera has been has hampered for clinical use, due to the low reproducibility between clinical batches [14].

The advent of technologies to generate monoclonal antibodies (mAbs) from mice and rats, which was first reported by Kolher and Milstein [16], has overcome many of the challenges associated with the use of human hyper-immune sera. OKT3, a mouse anti-human CD3, was the first mAb tested in human trials, and proven efficacious for prevention of organ rejection by inactivation of human T cells [17]. However, due to the mouse origin of OKT3 mAb, severe anaphylactic reactions were reported, and thereby the use of mAbs generated from animals has been discouraged for further clinical use [18].

In order to prevent anaphylactic reactions, technologies were aimed at genetic re-engineering of the animal mAb-Fc-backbone by human equivalents, although immune reactions against the mouse variable-Ig component could not be avoided [19]. A parallel approach involves the use of transgenic mice expressing part of the human Ig gene repertoire (XENOMOUSE™), which after immunization could be used to generate fully human mAbs [20]. Two licensed “fully human mAbs” generated using XENOMOUSE™ are panitumumab (Amgen, Washington D.C.) and denosumab (Amgen, Washington D.C.). Both products are currently being tested in human trials against colorectal cancer, and osteoporosis, respectively. Despite the success of XENOMOUSE™ for production of fully human mAbs, this approach still faces some challenges. First, the XENOMOUSE™ is proprietary (Abgenix, Fremont Calif.), preventing widespread public use. Secondly, the mouse B cells in XENOMOUSE™ have been shown to be going through a different maturation process from that of human B cells [20]. This raises questions as to the ability of XENOMOUSE™ to generate high-avidity, neutralizing mAbs against infectious agents. Furthermore, both panitumumab and denosumab are antibodies to human self-proteins (epidermal growth factor receptor (EGFR) and Receptor Activator of Nuclear Factor κB (RANK), respectively). Currently no neutralizing human mAbs against infectious diseases have been generated so far using the XENOMOUSE™. The use of human B cells from immunized-humans to generate human mAbs has been long envisioned as a strategy to avoid all the problems above mentioned. However, the generation of human mAbs using human B cells has been hindered by the very low frequency of antibody-producing human B cells in blood (10⁻⁴ to 10⁻⁶) [21] since activated B cells (plasma cells) are homed in lymphoid organs and bone marrow [22]. Consequently, while spleen biopsies have been shown as a very successful source of human B cells for generating human mAbs [23], the use of human peripheral blood as source of B cells has only led to a handful of successful attempts [24-26]. Furthermore, the successful attempts for generation of human mAbs using blood-derived human B cells requires prior transformation of B cells with Epstein Barr-virus, a relevant human pathogen, which imposes difficulties for their further development for clinical use.

The ability of new mouse model of this invention and that of the related application to generate fully human B cells that respond to vaccination provides a novel and unique resource to overcome all above mentioned challenges for product development. From these mice, we can obtain large numbers of antigen specific B cells from their lymphoid organs as sources for generation of human mAbs against infectious agents or toxin, such as liver and blood stage P. falciparum malaria. There has not been any successful attempts to generate “humanized” or “fully human” mAbs against human malaria parasites.

TABLE 2
Existing Mouse Models

| Mutate allele | Phenotype | Advantage | Disadvantage |
|---|---|---|---|
| NOD-scid KO | No mature T and B cells | low level of innate immunity | Residual innate immunity |
| | Radiation sensitive | low NK-cell function | Low but present NK-cell |
| | Decrease innate immunity | increased engraftment of | activity |
| | | human HSCs and PBMCs | Decrease lifespan owing to |
| | | | thymic lymphomas |
| NOD-scid KO, IL- | No mature T and B cells | Long lifespan | Lack appropriate MHC |
| 2Rγ KO | Radiation sensitive | Further reduction in innate | molecules for T-cell selection |
| | IL-2Rγ-chain deficiently; no | immunity | in the mouse thymus |
| | high-affinity signaling | NK cells absent | Seem to lack some human- |
| | through multiple cytokine | Higher level of engraftment of | specific cytokines required for |
| | receptors leading to many | human cells | human cell development and |
| | innate-immune defects | Develop functional human | survival |
| | | immune system | Low and variable level of T- |
| | | Complete absence of IL2rg | cell-dependant antibody |
| | | gene | responses |
| NOD-Rag1 KO | Rag1mutation leading to | Radiation resistant | Residual innate immunity |
| | lack of mature T and B cells | | Low but present NK-cell |
| | | | activity |
| | | | Low and Variable level of |
| | | | engraftment |
| NOD-Rag1 KO, | Rag1mutation leading to | Radiation resistant | |
| IL-2Rγ KO | lack of mature T and B cells | Long lifespan | |
| | IL-2Rγ-chain deficiently; no | Further reduction in innate | |
| | high-affinity signaling | immunity | |
| | through multiple cytokine | NK cells absent | |
| | receptors leading to many | Higher level of engraftment of | |
| | innate-immune defects | human cells | |
| | | Develop functional human | |
| | | immune system | |
| | | Complete absence of IL2rg | |
| | | gene | |
| NOD-scid, HLA- | Transgenic expression of | Long lifespan | Seem to lack some human- |
| A2, IL-2Rγ KO | human HLA-A2 | Further reduction in innate | specific cytokines required for |
| | No mature T and B cells | immunity | human cell development and |
| | Radiation sensitive | NK cells absent | survival |
| | IL-2Rγ-chain deficiently; no | Higher level of engraftment of | Low and variable level of T- |
| | high-affinity signaling | human cells | cell-dependant antibody |
| | through multiple cytokine | Develop functional human | responses |
| | receptors leading to many | immune system | |
| | innate-immune defects | Complete absence of IL2rg | |
| | | gene | |
| | | Transgenic expression of | |
| | | human MHC molecules | |
| BALB/c-Rag1 | Rag1 mutation leading to | Radiation resistant (can survive | Residual innate immunity |
| KO, | lack of mature T and B cells | high dose of radiation) | Low but present NK-cell |
| | | | activity |
| | | | Low and variable level of |
| | | | engraftment |
| BALB/c-Rag1 KO, | Rag1 mutation leading to | Radiation Resistant | |
| IL-2Rγ KO | lack of mature T and B cells | | |
| | IL-2Rγ-chain deficiently; no | | |
| | high-affinity signaling | | |
| | through multiple cytokine | | |
| | receptors leading to many | | |
| | innate-immune defects | | |
| DRAG | Rag1 mutation leading to | Develop human T and B cells | Restricted by mouse HLA |
| | lack of mature T and B cells | Reconstitute human IgG, IgM, | class I molecules |
| | No mature mouse T and B | IgA, IgE | |
| | cells | Respond to vaccination by | |
| | | eliciting human IgG response | |

### BRIEF DESCRIPTION OF THE FIGURES

FIG. 1. Generation of HLA-DR4 Tg, Abb KO, Rag2 KO mice (C57BL/6 background): Panel A, breeding strategy, Panel B, characterization of mice by FACS.

FIG. 2. Introduction of human CD80 (hCD80) transgene in HLA-DR4 Tg, Abb KO, Rag2 KO mice. Panel A shows expression of hCD80 by transfected BTC-6 insulinoma cells. Panel B shows PCR screening of littermates obtained by microinjection of RIP-hCD80 construct. Panel C, expression of hCD80 in pancreatic beta cells from hCD80 Tg littermates (right panel). No staining was detected in pancreatic beta-cells from non transgenic littermates (left panel).

FIG. 3. Generation of HLA-DR4 Tg, Rag1 KO, IL-2R γ KO (DRAG) and HLA-DR4 Tg, hCD80, Rag1 KO, IL-2Rγ KO (DRAG80) mice.

FIG. 4. Development of human T cells by DRAG mice. Groups of 4-6 week-old DRAG mice and control (Rag1 KO, IL2Rg KO) mice were irradiated (450 cGy) and infused intravenously with 40,000-80,000 human hematopoietic (CD34+) stem cells from HLA-DR4-positive umbilical cord bloods. Panel A, percentage of reconstituted mice. Panel B, Frequency of human T cells (CD3+) in blood. Panel C, frequency of CD4 T and CD8 T cell subsets.

FIG. 5. T cell response in DRAG mice. Splenic cells (3×10⁵) from DRAG or control mice were stimulated in vitro with anti-human CD3/CD28 Abs. As control, human PBMC (3×10⁵) were also stimulated with CD3/CD28 Abs.

FIG. 6. Development of regulatory CD4+FOXP3+ T cells (Tregs) by DRAG mice. Splenic cells from DRAG and control mice were stained with anti-human CD3, CD4, FOXP3 Abs and analyzed by FACS. Data shows frequency of Tregs in DRAG mice.

FIG. 7. Development of human B cells by DRAG mice. Groups of 4-6 week-old DRAG mice and control (Rag1 KO, IL2Rg KO) mice were irradiated (450 cGy) and infused intravenously with 40,000-80,000 human hematopoietic (CD34+) stem cells from HLA-DR4-positive umbilical cord bloods. Panel A, percentage of reconstituted mice. Panel B, Frequency of human B cells (CD19+) in blood. Panel C, levels of human IgM in blood, at several weeks post-infusion of human stem cells, Panel D, levels of human IgG in blood. Panel E left, identity of human proteins in sera of DRAG mice as determined by immunoelectrophoresis using human sera as comparator. Panel E right, no crossreactivity was observed in sera of non-reconstituted DRAG mice.

FIG. 8. IgG subclasses in DRAG mice. Levels of human IgG1, IgG2, IgG3, and IgG4 were measured in sera using ELISA kits (InVitrogen). Subclass distribution in two representative mice is shown.

FIG. 9. DRAG mice respond to TT vaccination. DRAG and control mice were immunized with TT vaccine and serum levels of anti-TT IgG were measured at day 14 post-immunization by ELISA. Data show that DRAG mice elicited anti-TT IgG antibodies whereas control mice failed to do so.

FIG. 10. Generation of humanized mice expressing HLA class I (A2.1) an HLA class II (DRB1*0401) molecules. Panel A, breeding strategy. Panel B, characterization of mice by FACS or PCR as described in methods.

FIG. 11. Expression of HLA-DR4 and HLA-A2 molecules in DRAG mice. Peripheral blood mononuclear cells from DRAGA (HLA-DR4.HLA-A2) mice or control (HLA-DR4-.HLA-A2-) littermates were stained with anti HLA-A2-PE and anti-HLA-DR-FITC antibodies and analyzed by FACS. Data show that DRAGA, but not control, mice express cells surface HLA-A2 and HLA-DR4 proteins.

### DETAILED DESCRIPTION OF THE INVENTION

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

### DEFINITIONS

The term “transgene” or Tg refers to the genetic material which has been or is about to be artificially inserted into the genome of an animal for protein expression, particularly a mammal and more particularly a mammalian cell of a living animal.

The term “knockout mutation” or KO refers to genetic material which has been or is about to be artificially inserted into the genome of an animal for disrupting endogenous genes and preventing protein expression.

“Transgenic (Tg) animal” refers to a non-human animal, usually a mammal, having a non-endogenous (i.e., heterologous) nucleic acid sequence present as an extrachromosomal element in a portion of its cells or stably integrated into its germ line DNA (i.e., in the genomic sequence of most or all of its cells) resulting in protein expression of the Tg. Heterologous nucleic acid is introduced into the germ line of such transgenic animals by genetic manipulation of, for example, embryos or embryonic stem cells of the host animal, and the Tg is perpetuated by breeding. Animals expressing various Tgs can be also generated by breeding parentals carrying each different Tgs

“Knockout (KO) animal” refers to a non-human animal, usually a mammal, having a non-endogenous (i.e., heterologous) nucleic acid sequence stably integrated into its germ line DNA (i.e., in the genomic sequence of most or all of its cells) to disrupt endogenous genes and prevent protein expression. Heterologous nucleic acid is introduced into the germ line of such KO animals by genetic manipulation of, for example, embryos or embryonic stem cells of the host animal, and the KO mutation is perpetuated by breeding. Animals expressing various KO mutations can be also generated by breeding parentals carrying each different KO mutations.

“Humanized mouse” refers to immune compromised mice engrafted with human haematopoietic stem cells or tissues, or mice that transgenically express human genes.

“Inbreeding” refers the mating of closely related individuals or of individuals having closely similar genetic constitutions.

“Intercross” refers to breeding from parents of different varieties or species.

“Crossbred” refers to an animal with purebred parents of two different breeds, varieties, or populations, often with the intention to create offspring that share the traits of both parent lineages, and producing an animal with hybrid vigor.

“Backcross” refers to mating the crossbred offspring of a two-way cross back to one of the parent breeds

“MHC” refers to Major Histocompatibility Complex antigens (also known as transplantation antigens) which are required for activation of antigen-specific immune cells. Among them, MHC class I refers to molecules required for activation of cytotoxic CD8 T cells. The MHC class I molecules are dimmers composed of an alpha (polymorphic, variable) chain and a beta-microglobulin (constant, non-polymorphic) chain. The MHC class II refers to molecules required for activation of helper/regulatory CD4 T cells. The MHC class II molecules are dimmers composed of an alpha (constant, non-polymorphic) and beta (polymorphic, variable) chain.

“HLA” refers to Human Leukocyte Antigens, which are MHC molecules only expressed by humans and required for antigen-specific activation of human immune cells. HLA-A2.1 refers to a particular HLA class I molecule consisting of the A2.1 alpha chain and beta2-microglobulin. HLA-DRB1*0401 (thereafter referred as HLA-DR4) refers to a particular HLA class II molecule consisting of the DRA1.1 alpha chain and B1*0401 beta chain.

“Human Costimulatory molecules” i.e., CD80 (also known as B7.1) are molecules required for enhancing human immune responses.

“Rag” refers to recombination activating gene, which encodes for a protein critical for development of mouse T and B cells.

“IL2Rgc” refers to interleukin 2 receptor gamma chain gene, which encodes for a protein critical for activation of mouse Natural Killer (NK, innate) immune cells.

HLA-A2.1 Tg (thereafter referred as HLA-A2) refers to transgenic animals expressing HLA-A2.1 molecules

HLA-DR*0401 Tg (thereafter referred as HLA-DR4) refers to transgenic animals expressing HLA-DR*0401

“Beta2-microglobulin (B2m) KO” refers to KO mice that do not express mouse endogenous MHC class I molecules.

“Abb KO” refers to KO animals that do not express endogenous mouse MHC class II molecules.

“CD80 Tg” refers to animals expressing transgenically the human costimulatory molecule CD80.

“Rag KO” refers to KO animals that cannot develop mouse T and B cells.

“IL2Rgc KO” refers to KO animals that cannot develop mouse NK immune responses.

This application relates to a transgenic animal model for testing immunogenicity and protective efficacy of human vaccines as well as a method for generating such a multi-transgenic animal. This application also relates to method for screening antigens and platforms for human vaccine development. More specifically, the present invention relates to a mouse model capable of co-expressing human leukocyte antigen DR4, and human leukocyte antigen A2.1, which upon infusion of human HLA-matched hematopoietic stem cells can develop a functional human immune system. This invention also discloses a method for producing fully humanized monoclonal antibodies for neutralization of infectious agents/toxins.

One aspect of the instant application is directed to a transgenic animal model whose genome comprises a nucleic acid construct comprising as least two transgenes each linked to a promoter effective for expression of human leukocyte antigen DR4 and human leukocyte antigen A2.1, and/or human costimulatory molecules (CD80), which can develop a functional human immune system upon infusion of human HLA-matched hematopoietic stem cells.

In one embodiment, at least two human genes were inserted into the genome of a genetically-altered mouse already containing a human gene, thereby producing a multi-transgenic mouse.

The genes introduced may include those coding for human leukocyte antigen DR4 (DR4 (B1*0401); Accession: AM698035.1), human leukocyte antigen A2.1 (A2.1; Accession: M17567.1) and/or human costimulatory molecules (CD80) (CD80; Accession: BC042665.1).

Another aspect of the instant application is directed to a process for producing a multi-transgenic animal in general. Typically, transgenic mice are generated by microinjecting a foreign gene into fertilized eggs isolated from a normal, nontransgenic mouse. In the instant invention, it has been shown that it is possible to create a mouse expressing additional human transgenes by starting with a mouse that is already transgenic. That is, single-cell embryos (fertilized eggs) from an existing transgenic mouse have been harvested, and additional transgene DNA fragments have been microinjected into the cells. The results shown herein demonstrate that existing fertilized eggs from transgenic mice can withstand the microinjection process to successfully produce a multi-transgenic mouse. In one embodiment, DNA fragments encoding for human CD80 is microinjected into a transgenic mouse already expressing human HLA-DR4 phenotype.

Yet another aspect of the instant application is the methods of producing fully human monoclonal antibodies (hMAb) and using them as a new therapeutic approach for neutralization of infectious agents/toxins. A method for producing a fully human antibody specific for a desired antigen, comprising: i) immunizing a transgenic mouse whose genome comprising a nucleic acid construct comprising as least one transgene linked to a promoter effective for expression of human leukocyte antigen DR4, which is capable of developing a functional human immune system upon infusion of human HLA-matched hematopoietic stem cells; and ii) recovering the antibody. The transgenic mouse may also express HLA-A2.1 or human CD80 as well as containing one or more mutations for knock out, including AbbKO, Rag KO, IL2RgcKO and B2m KO. Examples of the transgenic mouse may include DRAG, DRAGA, DRAGAB, DRAG80, DRAGA80 and DRAGAB80. The desired antigen is selected from the group consisting of: leukocyte markers; histocompatibility antigens; integrins; adhesion molecules; interleukins; interleukin receptors; chemokines; growth factors; growth factor receptors; interferon receptors; immunoglobulins and their receptors; tumor antigens; allergens; viral proteins; rickettsial proteins, bacterial proteins/glycoproteins; protozoal proteins/glycoproteins; helminth proteins/glycoproteins; toxins; blood factors; enzymes; ganglioside GD3, ganglioside GM2; LMP1, LMP2; eosinophil major basic protein, eosinophil cationic protein; pANCA; Amadori protein; Type IV collagen; glycated lipids; .gamma.-interferon; A7; P-glycoprotein; Fas (AFO-1) and oxidized-LDL. In one embodiment, humanized mice of this application expressing HLA molecules, such HLA-DR4 and/or HLA-A2.1 in a NOD.RagKO.IL2RgcKO background (DRAG or DRAG A mice) upon infusion of HLA-matched human hematopoietic stem cells, develop functional human B cells. These fully humanized B cells secrete specific IgG antibodies upon vaccination (PLoS One 6:e19826, 2011), and can be used as source of human B cells to generate “fully human” monoclonal antibodies used in therapies for neutralization of infectious agents or toxins.

### Example 1

### Generation of DRAG and DRAG80 Mouse Strains

Two mouse strains were genetically modified to sustain the development of the human hematopoietic system upon infusion of human stem cells. These strains are: Strain #1: HLA-DR4 Tg, Rag1 KO, IL2Rγ KO (NOD background), thereafter referred as DRAG mice. Strain #2: HLA-DR4 Tg, hCD80 Tg, Rag1 KO, IL2RγKO (NOD background), thereafter referred as DRAG80 mice.

Other mouse strains obtained during the process of generation of above strains are: Strain #3: HLA-DR4 Tg, Abb KO, Rag2 KO (C57BL/6 background). Strain #4: HLA-DR4 Tg, hCD80 Tg, Abb KO, Rag2 KO (C57BL/6 background)

TABLE 2
Outcome and Purpose of the Genetic
Modification of Mice Strain Used

| Genotype: | Outcome: | Purpose: |
|---|---|---|
| 1. Rag2 KO | Lack of mouse T and B | to prevent rejection of human |
| | cells | cells |
| 2. HLA-DR4 | Expression of human | to favor positive selection of |
| Tg | MHC II on thymic | human T cell precursors in |
| | stroma and peripheral | thymus and antigen-presentation |
| | APCs | in periphery |
| 3. hCD80 Tg | Expression of human | to sustain activation/survival |
| | costimulatory molecule | signals in mature T cells in |
| | CD80 (B7.1) | periphery |
| 4. Abb KO | Lack of endogenous | to prevent GvH by human T |
| | mouse MHC II mole- | cells |
| | cules | |
| 5. NOD | Permissive strain | favor HSC engraftment |
| background | | |
| 6. IL2Rγ KO | Lack of mouse NK | enhance HSC engraftment |
| | activity | |

Abbreviations: HLA-DR4: Human Leukocyte Antigen DR*0401; Rag2: recombination activation gene 2; Tg: transgenic; KO: knockout; Abb: mouse MHC class II (I-Ab)

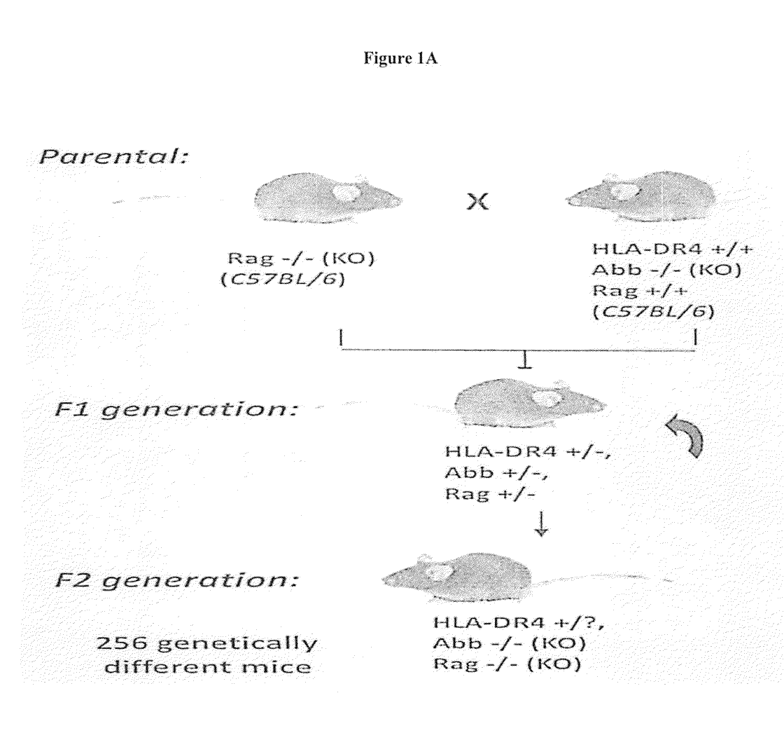

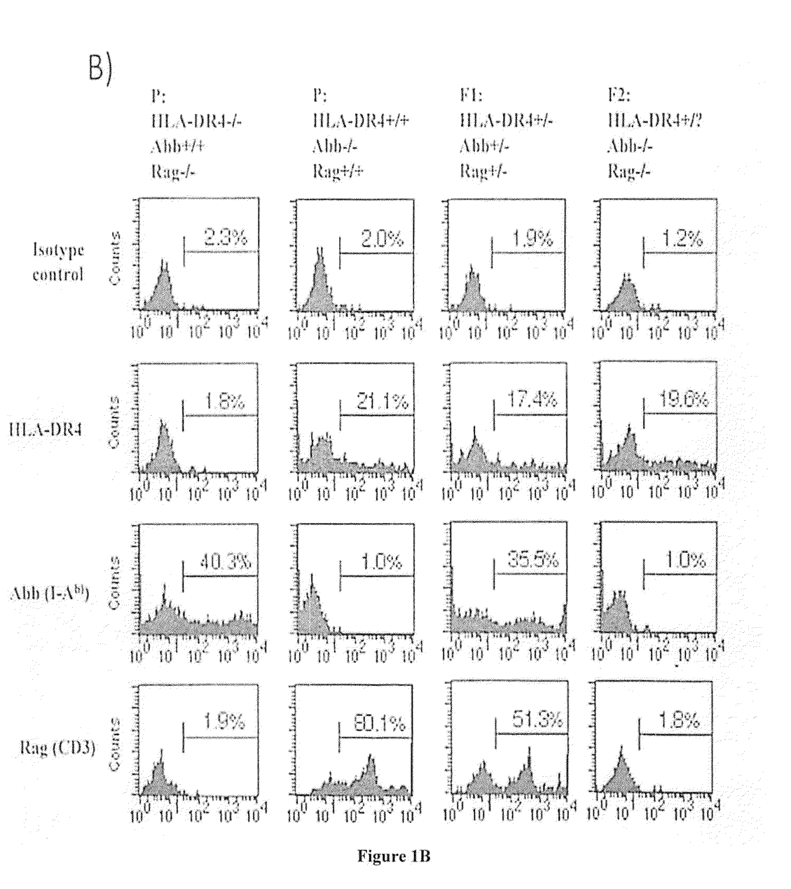

The DRAG and DRAG80 mouse strains were generated following four sequential steps that are illustrated in FIGS. 1-4.

### Step 1. Generation of HLA-DR4 Tg, Abb KO, Rag2 KO Mice in C57BL/6 Background

The new strain of mice (F2) was generated by breeding (FIG. 1). HLA-DR4 Tg, Abb KO mice in C57BL/6 background (line 4149) (Taconic Farms, Inc., New York), and Rag2 KO mice in C57BL/6 background (Taconic Farms, Inc., New York) were used as parental (P) mice to generate F1 mice that were heterozygous for all target genes of the parent mouse (HLA-DR4, Abb KO, and Rag2) (FIG. 1). F1 mice were intercrossed to generate F2 mice that were screened for expression of HLA-DR4 Tg and KO mutations for Rag2 and Abb loci. Screening of mice was performed by FACS analysis using specific antibodies and confirmed by PCR. FIG. 1, panel B shows F2 mice that expressing HLA-DR4 molecules, and KO for Abb and Rag2 genes were selected. This novel mouse strain F2 (HLA-DR4 Tg, Abb KO, Rag2KO in C57BL/6 background) is currently maintained at the WRAIR VetMed facility.

Step 2. Generation of HLA-DR4 Tg, hCD80 Tg, Abb KO, Rag2 KO Mice in C57BL/6 Background (M3)

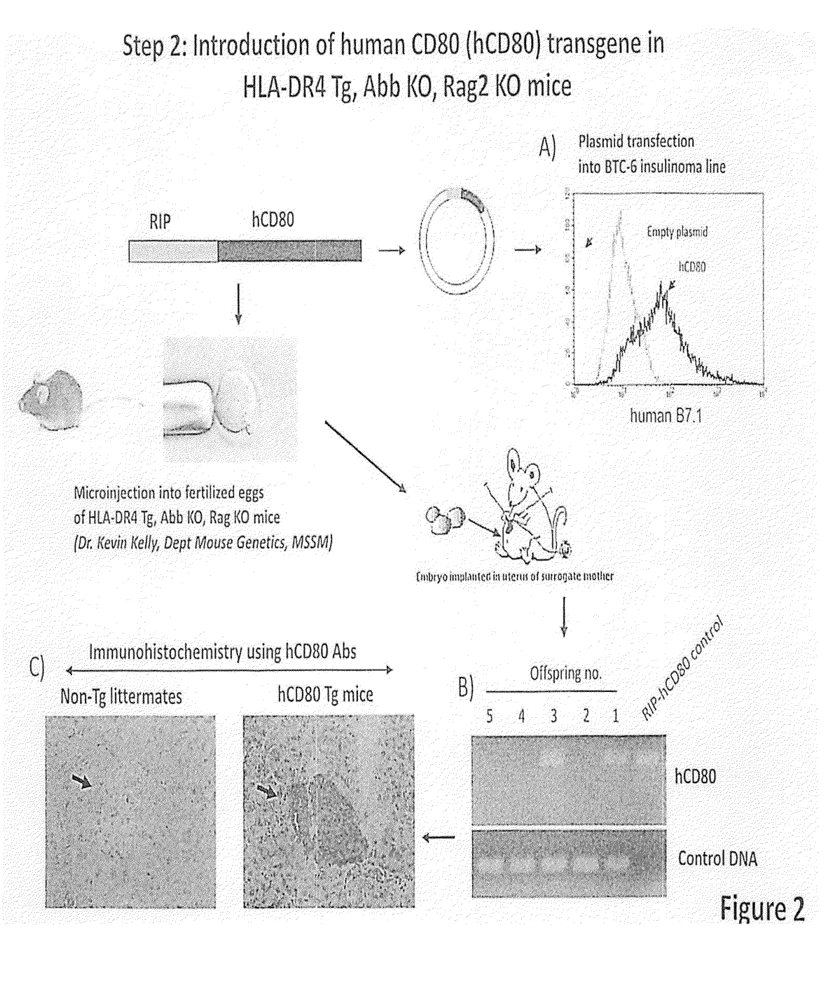

The M3 mouse strain was generated by microinjection of RIP-hCD8 DNA into fertilized eggs of F2 mice (HLA-DR4 Tg, Abb KO, Rag2 KO in C57BL/6 background) (FIG. 2).

Genetic Construction of RIP-hCD80:

The gene encoding for human CD80 (hCD80) was cloned by RT-PCR from total RNA extracted from Boleth (human B lymphoblastoma) cells. The genomic region of rat insulin promoter (RIP) was cloned by PCR from DNA extracted of splenic cells of RIP-HA Tg mice (a gift from Dr. Harald von Boehmer at INSERM, France) (11). Primers used for cloning of hCD80 gene and RIP contained restriction sequences that allowed the assembling of RIP and hCD80 in frame. To assess the structural integrity and the ability of the chimeric RIP-hCD80 genetic construct to express hCD80 protein, the RIP-hCD80 genetic construct was cloned in a pcDNA3 vector, and used to transfect BTC-6 mouse insulinoma cells. FIG. 2A demonstrated hCD80 expression on transfected cells.

Generation of HLA-DR4 Tg, hCD80 Tg, Abb KO, Rag2 KO Mice:

The RIP-hCD80 construct was introduced into fertilized eggs of HLA-DR4 Tg, Abb KO, Rag2 KO mice. The developed embryos were implanted in uterus of surrogate female dams, using standard procedures at the Mouse Genetics Core, Mount Sinai School of Medicine, New York, N.Y. Progeny was screened by PCR using specific primers for the RIP-CD80 genetic construct (FIG. 2B). Expression of hCD80 in pancreatic beta-cells from Transgenic littermates was assessed by immunohistochemistry using anti-hCD80 Abs. The results shown in FIG. 2C, right panel demonstrated expression of hCD80 Tg in pancreatic beta-cells. This new mouse strain M3 (HLA-DR4 Tg, hCD80 Tg, Abb KO, Rag2 KO) is currently maintained at WRAIR VetMed facility.

Step 3. Generation of DRAG Mice (HLA-DR4 Tg, Rag1 KO, IL2γ KO in NOD Background) and DRAG80 Mice (HLA-DR4 Tg, hCD80, Rag1 KO, IL2RγKO in NOD Background)

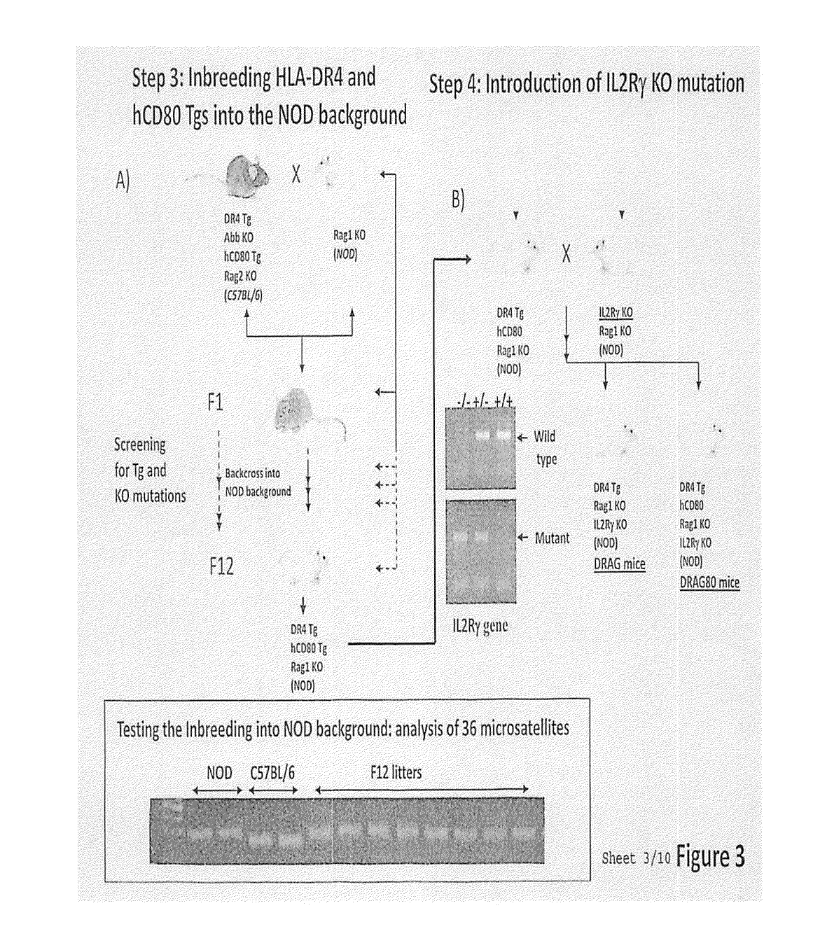

The DRAG and DRAG80 mouse strain were generated by inbreeding the HLA-DR4 and hCD80 Tg from the M3 mouse strain into the NOD/Rag1 background (FIG. 3A), followed by introduction of the IL2Rγ KO mutation into their genetic background (FIG. 3B)

FIG. 3A is a schematic representation showing the inbreeding the HLA-DR4 and hCD80 Tg into the NOD background. First, M3 mice (HLA-DR4 Tg, hCD80 Tg, Abb KO, Rag2 KO mice in C57BL/6 background) were backcrossed with Rag 1 KO mice in NOD background (The Jackson Laboratories, Bar Harbor, Me.) for twelve generations. For each generation, mice were screened for HLA-DR4 and hCD80 Tgs and Rag 1 KO mutations.

To determine the level of inbreeding into the NOD background, the progeny from F12 generation were examined for 36 microsatellite regions by PCR (FIG. 3A, lower panel). F12 mice showed complete microsatellite identity with NOD for the 36 microsatellite regions.

Next, the HLA-DR4 Tg, hCD80 Tg, Rag1 KO (NOD) mice were crossed with IL2RγKO, Rag1 KO (NOD) mice (The Jackson Laboratories, Bar Harbor, Me.) (FIG. 3B). Heterozygous F1 mice were intercrossed to generate F2 mice, and the F2 mice were examined by PCR for HLA-DR4 and hCD80 Tgs and IL2RγKO mutations (FIG. 3B). From F2 mice, mice having the genotype HLA-DR4 Tg, Rag1 KO, IL2Rγ KO (strain #1, DRAG), and mice having the genotype HLA-DR4 Tg, hCD80 Tg, Rag1 KO, IL2RγKO (strain #2, DRAG80) were selected. These new mouse strains are currently maintained at the WRAIR VetMed Facility.

### Example 2

### Functionality Testing of DRAG and DRAG80 Transgenic Mouse

The newly-developed humanized mice DRAG (HLA-DR4 Tg, Rag1 KO, IL2RγKO and DRAG80 (HLA-DR4 Tg, hCD80, Rag1 KO, IL2R KO DRAG80) are superior than the current humanized mouse strains (NOD-scid KO; NOD-scid KO, IL2Rγ KO; NOD-Rag1 KO, NOD-Rag1 KO IL2Rγ KO; BALB/c-Rag1 KO; and BALB/c-Rag1 KO, IL2R IL2Rγ KO). They are capable of:

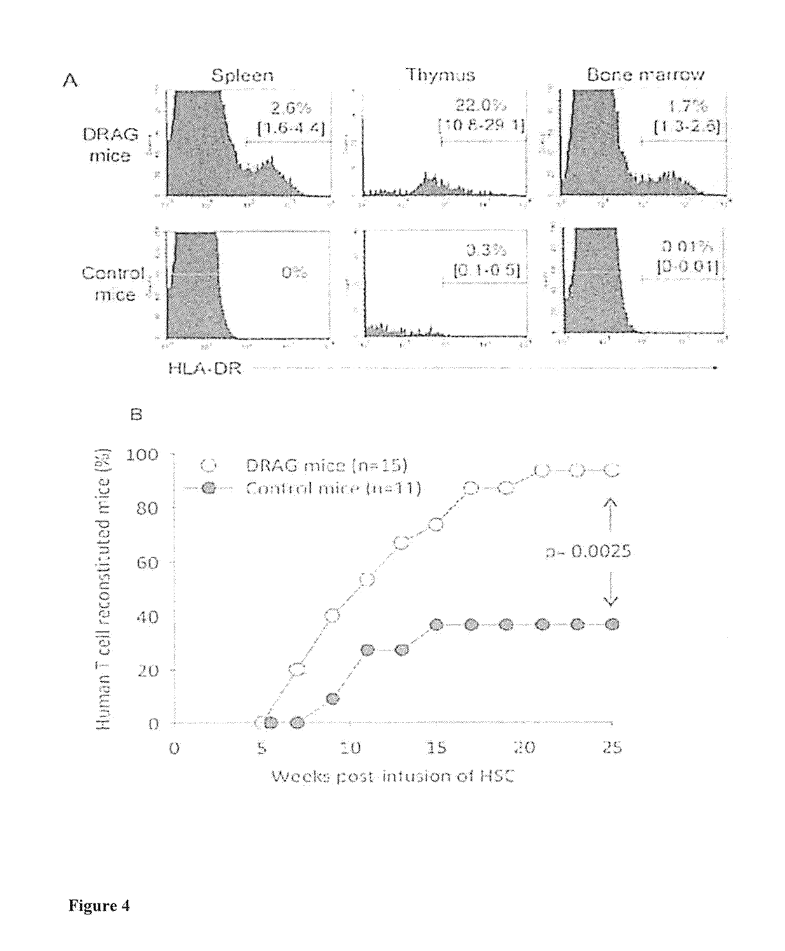

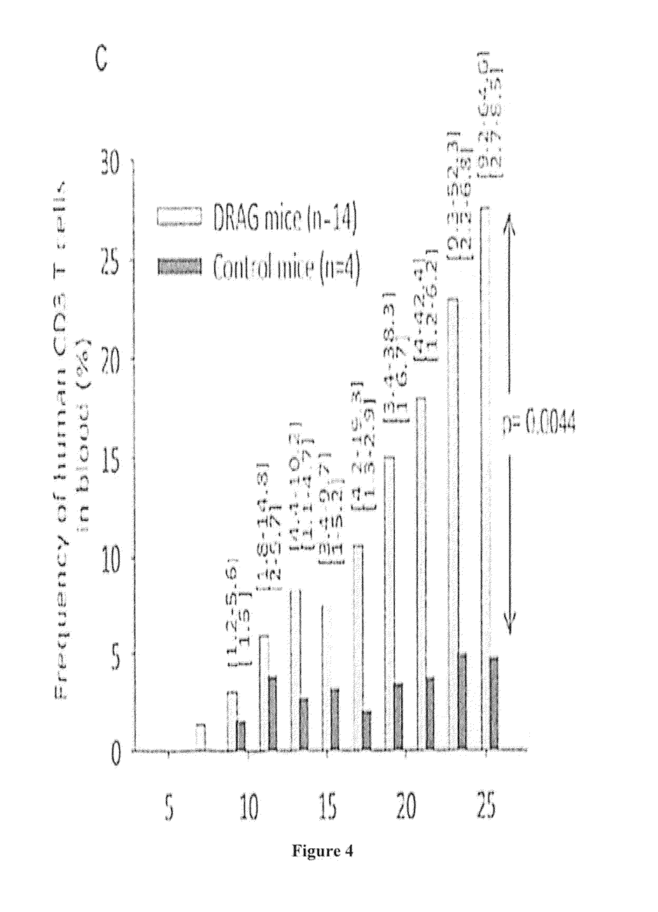

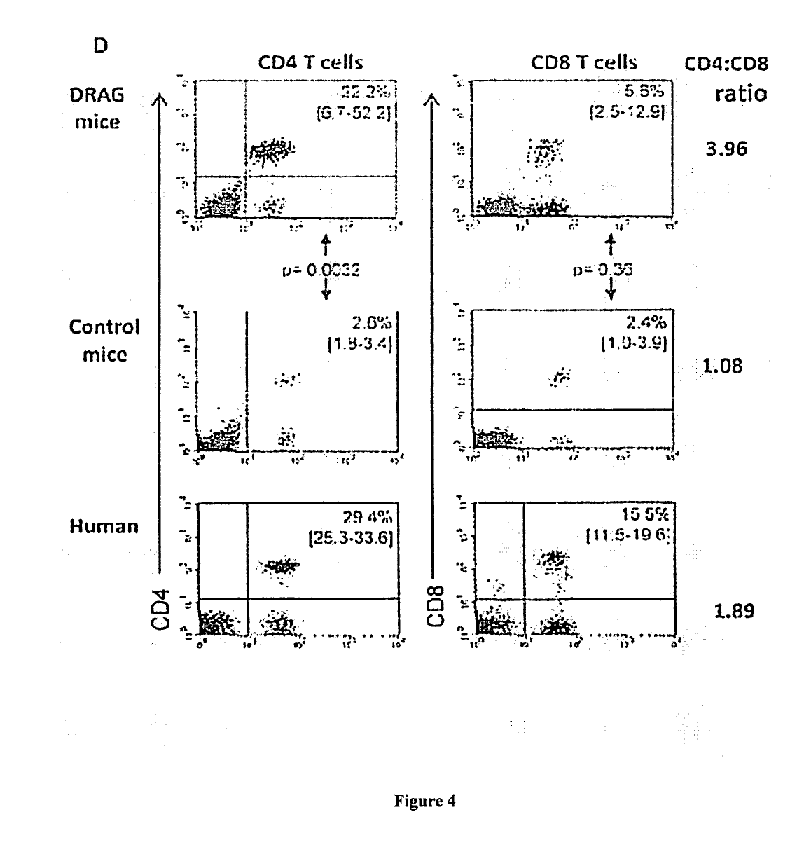

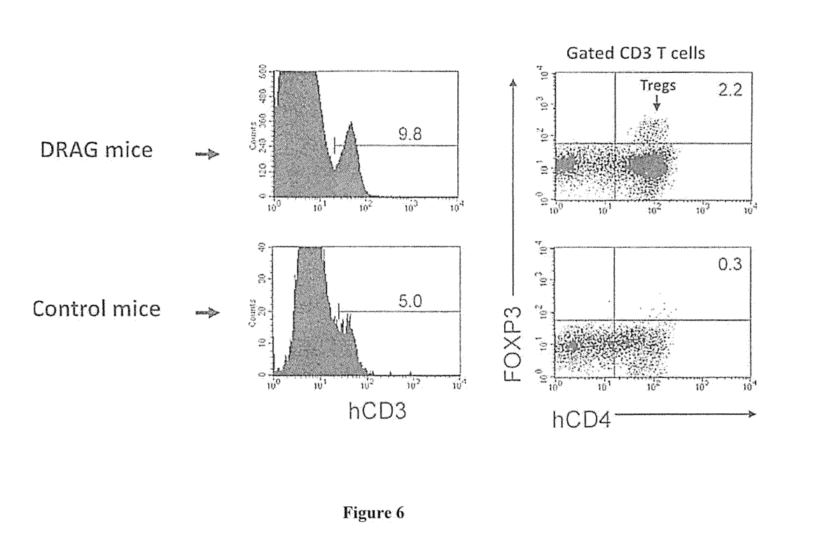

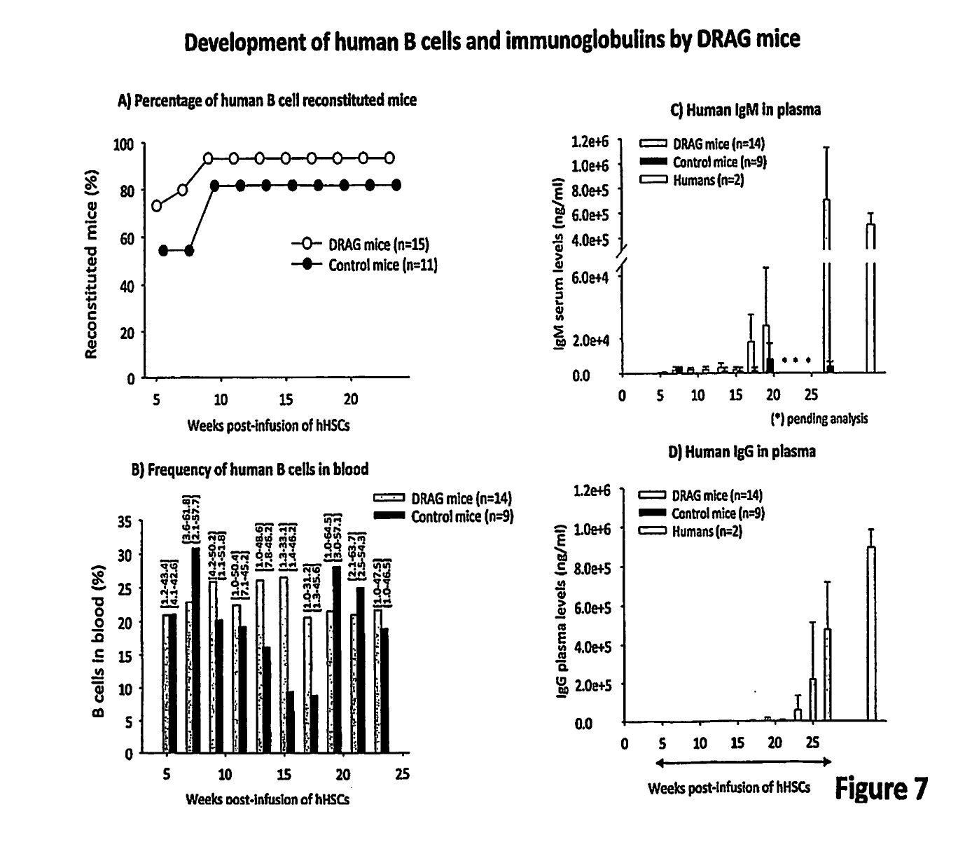

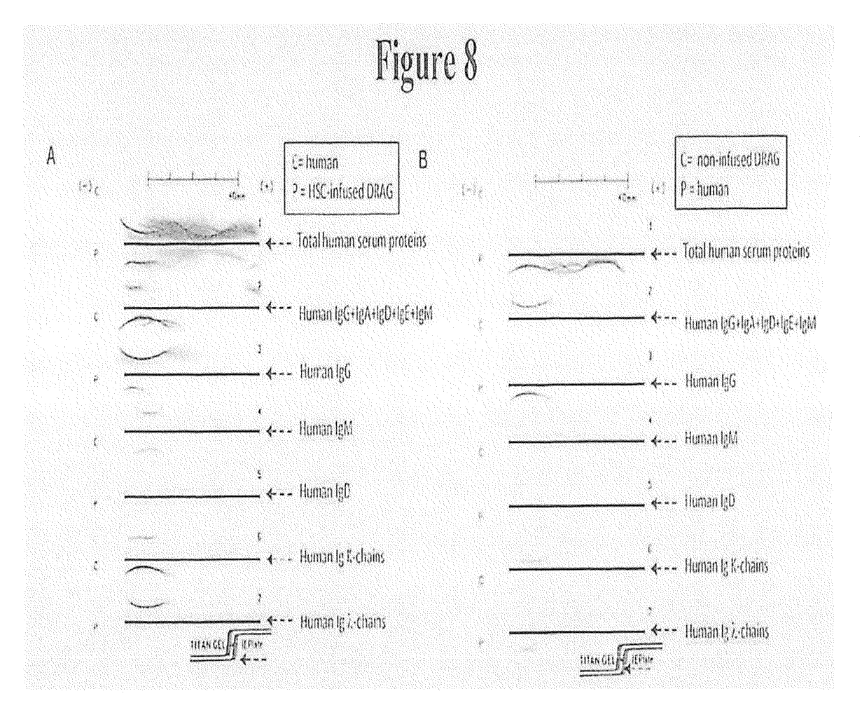

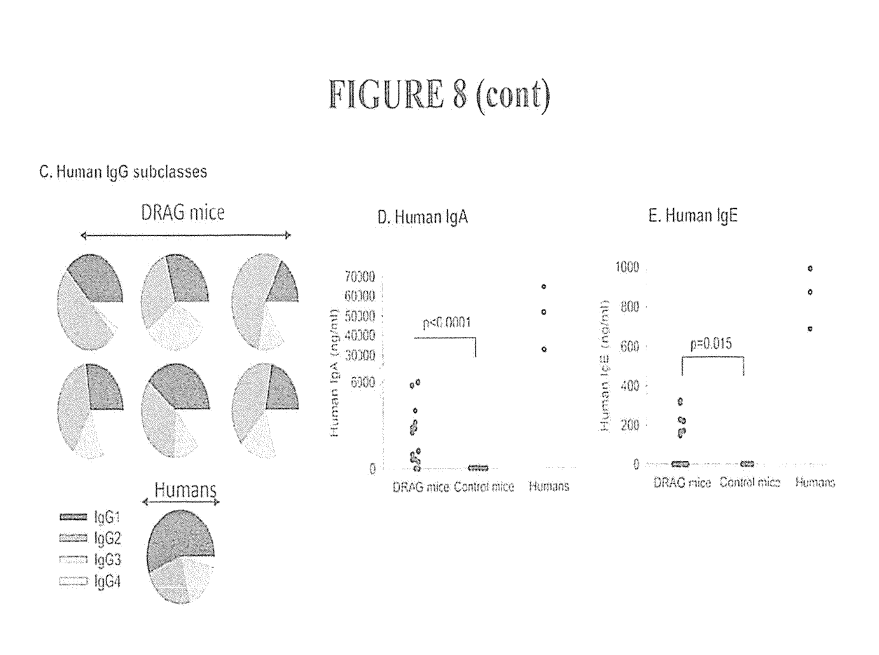

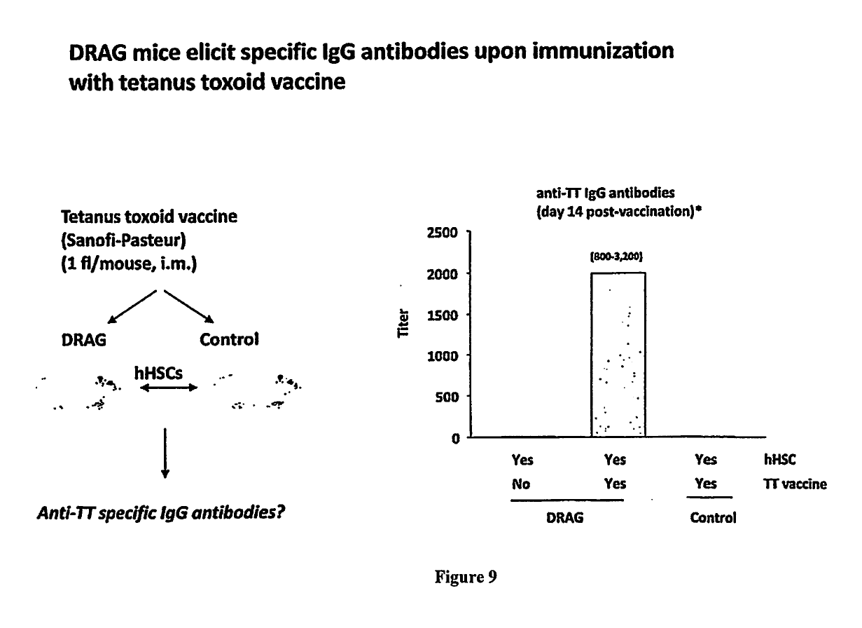

1) providing high level of human T cell reconstitution and frequencies of CD4 T and CD8 T cell subsets (FIG. 4)
2) allowing development of human regulatory CD4+FOXP3+ T cells (FIG. 6)
3) allowing development of serum levels of human IgM and IgG comparable to human blood (FIG. 6C, FIG. 7)
4) allowing development of human IgG1, IgG2, IgG3, IgG4 subclasses (FIG. 8) and
5) providing the ability to elicit specific IgG antibodies upon vaccination (FIG. 9)

To test the functionality of the transgenic mouse of the present invention, 4-6 week old mice were irradiated at 450 cGy, and injected intravenously with 40,000-80,000 human hematopoietic stem cells purified from umbilical cord blood of newborns of HLA-DR4 (0401) haplotype.

The mice tested were DRAG mice (HLA-DR4 Tg, Rag1 KO, IL2Rγ KO in NOD background). As control, Rag1 KO, IL2Rg KO littermates (negative for HLA-DR4 and hCD80 Tgs) were used. The control mice are identical to the commercial strain NOD-Rag1 KO, IL2Rγ KO. Their use allowed a strict comparison between the control and the test mouse models. Previous studies demonstrated no significant difference between NOD-Rag1 KO, IL2R littermates and the other commercial strains in terms of development of a human immune system (3, 4).

### Results

### Higher Level of Human T Cell Reconstitution and Frequencies of CD4 T and CD8 T Cell Subsets in DRAG Mice as Compared to Control Mice.

Pre-T cells derived from differentiation of hematopoietic stem cells in the bone marrow migrate to the thymus to undergo either positive (survival) or negative (deletion) selection upon recognition of self-peptides presented by thymic stromal cells in the context of MHC molecules. This is a physiological process aimed at preventing autoimmunity, by means of elimination of T cells that can react against self-antigens. T cell precursors in thymus that are positively selected receive survival signals that enable them to migrate and repopulate peripheral lymphoid organs (12).

The current humanized mouse models do not express human MHC (HLA) molecules and consequently the differentiation of T cells is thought to occur extra-thymically. Extra-thymic differentiation of human T cells in the current humanized mouse models may thus account for poor human T cell development and function, as it has been widely reported (2-9). DRAG mice express transgenically HLA-DR4 molecules and consequently they are expected to allow thymic differentiation of T cell precursors derived from HLA-matched hHSCs.

To test the hypothesis, groups of DRAG mice and control mice were injected with hHSC of HLA-DR4 haplotype, and they were followed for development of human T cells in peripheral blood by FACS. These analyses were performed at various time points upon hHSC infusion. As illustrated in FIG. 4A, most of DRAG mice (14 out 15) developed human T cells as compared to only 4 out of 11 control mice when infused with hHSCs from the same donors. Furthermore, the frequency of human T cells in reconstituted DRAG mice was significantly higher than in those control mice that developed human T cells (FIGS. 4B and 4C).

### Development of Functional Human T Cells

To assess the function of human T cells developed by DRAG, groups of mice (both DRAG and control mice) were euthanized and splenic T cells were stimulated with human CD3/CD28 antibodies. Stimulation with CD3/CD28 antibodies is a common approach to test selectively the function of T cells.

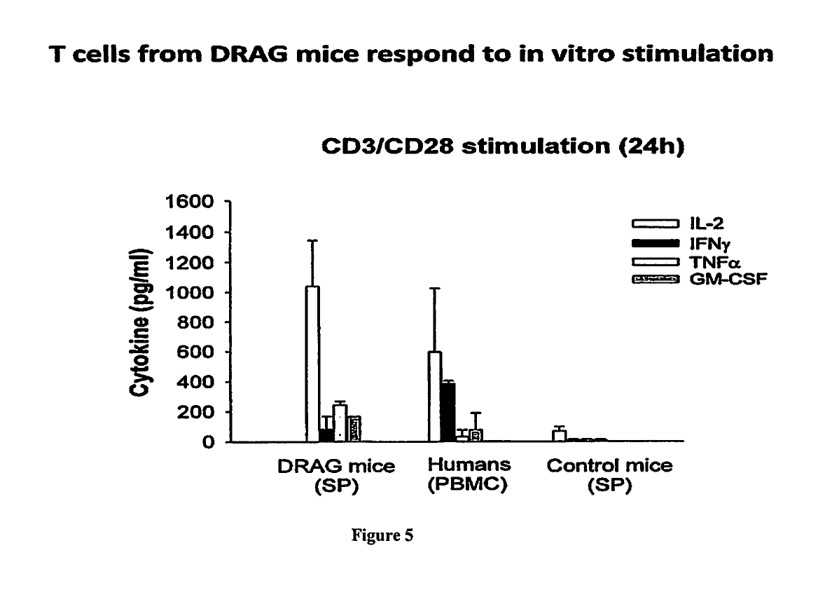

As illustrated in FIG. 5, the T cells from DRAG mice, but not T cells from control mice, responded vigorously to CD3/CD28 stimulation, as measured by the level of cytokines secreted in cell culture supernatants. The lack of T cell response in control mice is in agreement with previous studies indicating that T cells from stem cell-reconstituted NOD-scid respond poorly to CD3/CD28 stimulation in vitro (3). On the other hand, T cell response of DRAG mice was similar to that of T cells from human blood. This result demonstrated that T cells from DRAG mice are fully functional, whereas the T cells from control mice are impaired in function.

### Development of Human Regulatory CD4+FOXP3+ T Cells

Regulatory T cells, particularly those from the CD4+FOXP3+ subset (Tregs), are an important compartment of the immune system, whose function is to maintain self-tolerance in periphery and to down-regulate aggressive immune responses to pathogens once the infection has been cleared (13). Development of Tregs by the current humanized mouse model has not been reported in the literature. However, as illustrated in FIG. 6, DRAG mice developed CD4+FOXP3+ Tregs, whereas the control mice failed.

These test results demonstrated that the human immune system developed by DRAG mice comprises all T cell compartments as developed by humans.

### Development of Serum Levels of Human IgM and IgG Comparable to Human Blood

The function of B cells is to secrete antibodies (immunoglobulins) that are mainly found in blood and body fluids. The function of immunoglobulins is to counterattack pathogens such as viruses, bacteria, and parasites as well as to control the homeostatic growth of commensal bacteria in the intestinal tract (reviewed in 14). In mammalians there are five classes of immunoglobulins, namely IgD, IgM, IgG, IgA, and IgE.

In physiological conditions, IgD is found only on the surface of B cells. Upon antigen encountering, B cells undergo a rearrangement at the DNA level to switch antibody class from IgD to a secretory form of IgM. Fully functional B cells then undergo a further DNA rearrangement to switch class from IgM to either IgG, IgA, or IgE. The IgG class is by far the most abundant immunoglobulin in blood.

Both DRAG mice and control showed similar level of reconstitution (FIG. 7A) and frequency of human B cells (FIG. 7B). However, DRAG mice produce significantly higher levels of human IgM than control mice (FIG. 7C). Furthermore, the levels of serum IgM in DRAG mice were comparable to those in human blood. More importantly, DRAG mice produced serum levels of human IgG, whereas control mice failed to produce human IgG (FIG. 7D). Identity between human immunoglobulins developed by DRAG mice and immunogobulins in human sera was compared by immunoelectrophoresis (FIG. 7E). The results demonstrate that human B cells developed by DRAG are fully functional whereas those from control mice are impaired in their ability to produce human immunoglobulins and undergo class switch from IgM to IgG.

### Development of Human IgG1, IgG2, IgG3, IgG4 Subclasses

In humans, IgG comprises four different subclasses that differ in biological functions namely (i) ability to cross placenta, (ii) activate the complement cascade and (iii) bind to Fc receptors expressed on phagocytic cells (15). As illustrated in FIG. 8, DRAG mice elicited all four subclasses of human IgG. These data further demonstrated that human B cells developed by DRAG are as functional as B cells developed by humans.

### Ability to Elicit Specific IgG Antibodies Upon Vaccination

The function of the immune system is to protect against infections by eliciting specific immune responses able to counterattack pathogens. Vaccines are biological preparations that have been used for centuries to stimulate the immune system in order to provide immunity to a particular infectious agent (16).

Groups of DRAG mice and control mice were immunized with tetanus toxoid (TT) vaccine (a vaccine currently used for humans) and 14 days post-vaccination, the serum levels of anti-TT human IgG antibodies were measured. As illustrated in FIG. 9, DRAG mice elicited anti-TT IgG antibodies in titers ranking from 800 to 3,200. In contrast, control mice failed to produce anti-TT IgG antibodies. The results demonstrate that DRAG mice developed a functional human immune system able to elicit specific immune responses upon vaccination.

### Prophetic Example 3

### Functionality Test for DRAG80

Data shows expression of human CD80 in transfected cells as indicated by FACS using anti-CD80 antibodies. The RIP-CD80 genetic construct was injected into fertilized eggs of HLA-DR4 Tg mice, and two independent CD80 Tg lines were obtained. The CD80 transgenic mice showed expression of CD80 protein as indicated by immunohistochemistry in their pancreas using anti-CD80 antibodies. Experiments similar to those contained in Example 2 are also conducted to furthered test the functionality of DRAG80.

### Example 4

### Generation of DRAGA Mouse Model

A mouse model (DRAGA) capable of co-expressing human leukocyte antigen DR4, and human leukocyte antigen A2 was genetically modified to sustain the development of the human hematopoietic system upon infusion of human stem cells.
Strain #1: HLA-DR4.HLA-A2.1.RagKO.IL2RgcKO in NOD background; thereafter referred as DRAGA mice.

Other mouse strains obtained during the process of generation of above strains are:
Strain #2: HLA-DR4.HLA-A2.1.hCD80.RagKO.IL2RgcKO in NOD background; thereafter referred as DRAGA80 mice;
Strain #3: HLA-DR4.HLA-A2.1.B2mKO.RagKO.IL2RgcKO in NOD background; thereafter referred as DRAGAB mice; and
Strain #4: HLA-DR4.HLA-A2.1.hCD80.B2mKO.RagKOIL2RgcKO in NOD background; thereafter referred as DRAGAB80 mice.

In an embodiment, the transgenic mouse capable of co-expressing HLA-DR4 and HLA-A2.1 upon infusion of human HLA-matched hematopoietic stem cells is created in following steps:

Step 1. Generation of F1 Hybrids Mouse Strains with Genotype HLA-DR4+/−,HLA-A2.1+/−.hCD80+/−.B2 m+/−Rag1+/−,IL2Rgc+/− in NOD Background

DRAG80 mice (HLA-DR4.hCD80.Rag1KO.IL2RgcKO.NOD) have been previously disclosed (PCT/US11/001,656) and mice with HLA-A2.1.B2m−/− in NOD background were purchased from the Jackson Labs (The Jackson Laboratories, Bar Harbor, Me.). The DRAG80 mice and HLA-A2.B2m−/− were crossed to generate F1 hybrids with the following genotype HLA-DR4+/−,HLA-A2+/−.hCD80+/−.B2 m+/−Rag+/−,IL2Rgc+/− in NOD background.

### Step 2. Generation of DRAGA Mouse Strain

The F1 hybrid mice were intercrossed and the F2 litters were screened for the different genes as described in Example 2 and PLoS ONE 6: e19826. Rag KO mutation is confirmed by FACS using peripheral blood cells stained with anti-mouse CD3 Antibodies. HLA-DR4 transgene is confirmed by PCR using primers as described previously and in PLoS ONE 6: e19826, 2011. HLA-A2.1 transgene is confirmed by PCR using primers forward TCCATGAGGTATTTCTTCAC (SEQ ID NO.: 5) and reverse GGCCTCGCTCTGGTTGTAG (SEQ ID NO.: 6). IL2Rgc KO mutation is confirmed by PCR using primers as described in PLoS ONE 6: e19826, 2011. B2m KO mutation is confirmed by PCR using primers B2m-COMMON-Forward TAT CAG TCT CAG TGG GGG TG (SEQ ID NO.: 7) and B2m-Mutant-Reverse primer TCT GGA CGA AGA GCA TCA GGG (SEQ ID NO.: 8). The presence of wild type B2m allele was detected by PCR using B2m-COMMON-Forward primer and B2m-WildType-Reverse primer CTG AGC TCT GTT TTC GTC TG (SEQ ID NO.: 9). hCD80 transgene is confirmed by PCR using primers as described in previous section (Example 1).

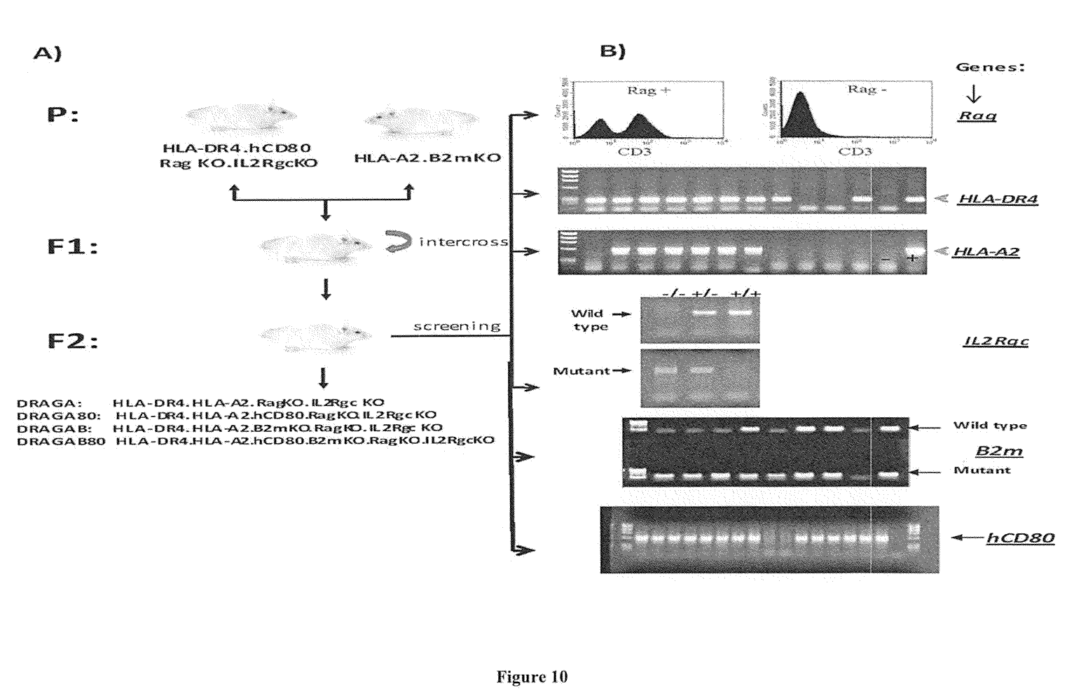

Among the F2 litters, four new humanized mouse strains were obtained with the following genotype (FIG. 10):

Strain #1, DRAGA mice: HLA-DR4.HLA-A2.1.Rag1KO.IL2RgcKO.NOD;
Strain #2, DRAGA80 mice: HLA-DR4.HLA-A2.1.hCD80.Rag1KO.IL2RgcKO.NOD;
Strain #3, DRAGAB mice: HLA-DR4.HLA-A2.1B2mKO.Rag1KO.IL2RgcKO.NOD;
Strain #4, DRAGAB80 mice: HLA-DR4.HLA-A2.2.hCD80.B2mKO.Rag1KO.IL2RgcKO.NOD

### Example 5

### Functionality Testing of Transgenic Mouse DRAGA

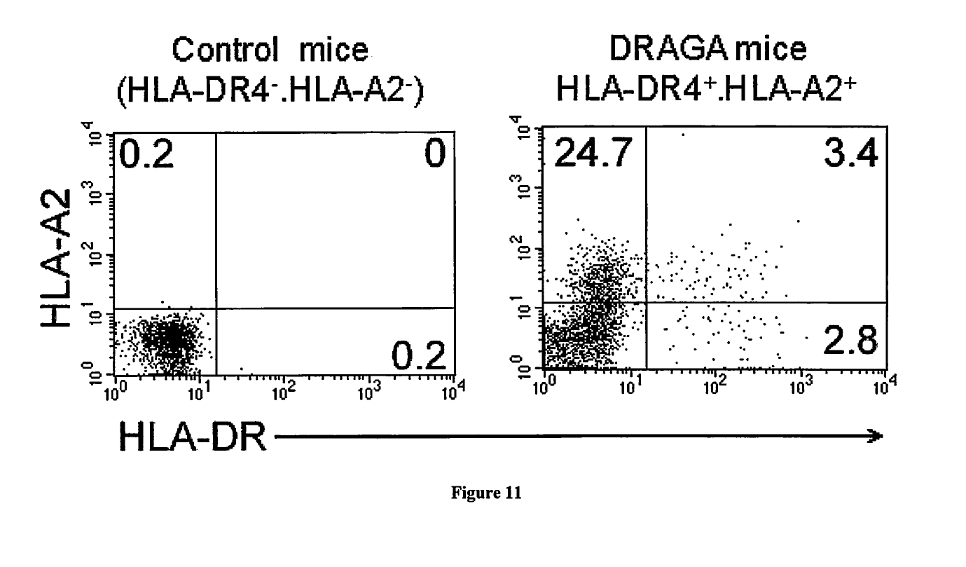

Expression of HLA-DR4 and HLA-A2 molecules in DRAGA mice is shown in FIG. 11. The results indicated that both transgenes are expressed as proteins on cell surface and they are recognized by specific antibodies. The advantages of the newly-developed humanized mouse strains rely on co-expression of HLA class I and class II molecules, which upon infusion of HLA-matched human hematopoietic stem cells results in development of human CD4 T and CD8 T cells that are restricted by human HLA molecules. Due to the human HLA restriction, these novel humanized mouse strains allow determining immunogenicity and protective efficacy of human vaccines.

The strains 1-4 is also confirmed as capable of reconstituting human T cells (including regulatory CD4+FOXP3+ T cells), B cells, dendritic cells and serum levels of human IgM, IgG, IgGA, and IgE similarly to DRAG mice using procedures as described for functionality tests of DRAG mouse model and PLoS ONE 6: e19826, 2011.

The transgenic mouse of this invention may be used in a method for evaluating an agent for human vaccine. An embodiment of this method comprising 1) providing two set groups of same strain transgenic mouse, including but not limited to DRAG, DRAGA, DRAG80 or DRAGAB; 2) administering an agent to one said group of transgenic mouse, comparing the immunogenic response of this group of transgenic mouse with the immunogenic response of the other group of transgenic mouse to which no agent has been administered, wherein an agent that induces a higher immunogenic response is identified as an agent for potential vaccine use. The immunogenic response maybe a humoral immune response or a cellular immune response. The agent maybe biologics, pharmaceuticals, or chemicals. The agent maybe administered with a pharmaceutically acceptable carrier or an adjuvant.

### Prophetic Example 6

### Testing DRAGA Mice and Derivates' Ability to Elicit HLA-A2.1-Specific Cytotoxic Cells Upon Immunization

Upon immunization with a peptide from influenza A virus which is presented in the context of HLA-A2.1 molecules, strains 1-4 (DRAGA, DRAGA80, DRAGAB, DRADAB80) developed cytotoxic human CD8 T cells that are capable to kill target cells in vitro. In contrast, the human CD8 T cells from DRAG and DRAG80 mice (lacking expression of HLA-A2.1) were unable to kill target cells. The results demonstrated that like humans, the newly-generated mouse strains respond to vaccination by eliciting antigen specific (HLA-A2 restricted) human cytotoxic CD8 T cells. Strains 1-4 also elicit cytotoxic CD8 T cells in vivo able to clear pathogens and prevent infection.

### Prophetic Example 7

### Generate Therapeutic Human Monoclonal Antibodies to Infectious Agent Using DRAG Mice as Source of Human B Cells

For generation of human mAbs against P. falciparum sporozoites/liver stage parasites, groups of DRAG mice (n=7) will be immunized with irradiated P. falciparum sporozoites (10,000 rads, three doses of 10⁵ at two weeks apart). To generate human mAbs against P. falciparum blood stage parasites, groups of mice will be injected intravenously with infected-red blood cells under chloroquine cover, as this approach has been shown to confer protection against blood stage infection [19].

Two weeks after immunization, mice sera will be tested for antibodies to P. falciparum sporozoites, liver stage parasites (using P. falciparum-infected HC04 cells), and infected-red blood cells by IFA. Mice eliciting specific antibodies will be euthanized, and their splenic, lymph-node, and bone-marrow human B cells will be fused with K6H6/B5 (an immortal human myeloma cell line that does not secrete human Igs, ATTC) using standard PEG-based protocols (7,8), and hybridoma cells will be cloned by limiting dilution using standard procedures. Supernatants from growing hybridomas will be tested for specificity to the above mentioned parasites by IFA, and ELISA. Biological activity will be measured by in vitro inhibition of parasite growth. REFERENCES
1 Shultz, et al. Humanized mice in translational biomedical research, Nat. Rev. Immunol. 7 (2007)
2 Heuts et al. Alloreactivity but failure to reject islet transplants by humanized BALB/c rag2gc mice. Scand J Immunol. 71: 83-90, 2010
3 Watanabe et al. The analysis of the functions of human B and T cells in humanized NOD/shi-scid/gammanull (NOG) mice (hu-HSC-NOG). Int. Immunol. 21:843-58, 2009
4 Brehm et al. Parameters for establishing humanized mouse models to study human immunity: analysis of human hematopoietic stem cells engraftment in 3 immunodeficient strains of mice bearing the IL2Rgamma (null) mutation. Clin. Immunol. 135:84, 2010
5 Lent et al. IL-7 enhances thymic human T cell development in “human immune system” Rag2−/−IL2Rgammac−/− mice without affecting T cell homeostasis. J, Immunol. 183:7645-55, 2009
6 Jaiswal et al. Dengue Virus Infection and Virus-Specific HLA-A2 Restricted Immune Responses in Humanized NOD-scid IL2rcnull Mice. PLOS One 4:e7251, 2010
7 Ishikawa et al. Development of functional human blood and immune systems in NOD/SCID/IL2 receptor {gamma} chain(null) mice. Blood 106: 1565-1573, 2005
8 Sato et al. Dynamics of memory and naïve CD8+ T lymphocytes in humanized NOD/SCID/IL-2Rgammanull mice infected with CCR5-tropic HIV-1. Vaccine 28 suppl 2:B32-7, 2010
9 Kwant-Mitchell et al. Mucosal innate and adaptive immune responses against herpes simplex virus type 2 in a humanized mouse model. J, Virol. 83:10664-76, 2009.
10 Casares, S. & Brumeanu, T. D. Insights into the pathogenesis of type 1 Diabetes: a hint for novel immunospecific therapies. Curr. Mol. Med. 1:357-378, 2001.
11 Strowig T, Gurer C, Ploss A, Liu Y F et al. Priming of protective T cell responses against virus-induced tumors in mice with human immune system components. J Exp Med. 206(6):1423-34. (2009).
12 Shultz L D, Saito Y, Najima Y, Tanaka S. et al. Generation of functional human T-cell subsets with HLA-restricted immune responses in HLA class I expressing NOD/SCID/IL2r gamma(null) humanized mice. Proc Natl Acad Sci USA. 2010 Jul. 20; 107(29):13022-7.
13 Danner R, Chaudhari S, Rosenberger J, Surls J, Richie T L. Brumeanu T D & Casares S. Expression of HLA class II molecules in humanized NOD.Rag1KO.IL2RgcKO mice is critical for development and function of human T and B cells. PLoS ONE 6: e19826 (2011).
14 Alves-Rosa F, Vermuelen M, Cabrera J, StanganelliC, Capozzo A, Narbaitz M, van Rooijen N, Palermo M. Macrophage depletion following liposomal-encapsulated clodronate (LIP-CLOD) injection enhances megakaryocytopoietic and thrombopoietic activities in mice. British J. Haematology 121:130-138 (2003)
15 Von Behring E, Kitasato S (1991) The mechanism of diphtheria immunity and tetanus immunity in animals. Mol Immunol 28: 1317, 1319-1320.
16 Kohler G, Milstein C (1975) Continuous cultures of fused cells secreting antibody of predefined specificity. Nature 256: 495-497.
17 Baker M (2005) Upping the ante on antibodies. Nat Biotechnol 23: 1065-1072.
18 Scott C T (2007) Mice with a human touch. Nat Biotechnol 25: 1075-1077.
19 Presta L G (2008) Molecular engineering and design of therapeutic antibodies. Curr Opin Immunol 20: 460-470.
Jakobovits A, Amado R G, Yang X, Roskos L, Schwab G (2007) From XenoMouse technology to panitumumab, the first fully human antibody product from transgenic mice. Nat Biotechnol 25: 1134-1143.
21 Stevens R H, Macy E, Morrow C, Saxon A. Characterization of a circulating subpopulation of spontaneous antitetanus toxoid antibody producing B cells following in vivo booster immunization. J Immunol 1979; 122:2498.
22 Review on homing
23 Olsson L, Kaplan H S. Human-human hybridomas producing monoclonal antibodies of predefined antigenic specificity. Proc Natl Acad Sci USA 1980; 77:5429.
24 Xiaocong Yua, Patricia A. McGrawa, Frances S. Housea, and James E. Crowe Jr An optimized electrofusion-based protocol for generating virus specific human monoclonal antibodies J Immunol Methods. 2008 Jul. 31; 336(2): 142-151.
25 Traggiai E, Becker S, Subbarao K, Kolesnikova L, Uematsu Y, et al. (2004) An efficient method to make human monoclonal antibodies from memory B cells: potent neutralization of SARS coronavirus. Nat Med 10: 871-875.
26 Kwakkenbos M J, Diehl S A, Yasuda E, Bakker A Q, van Geelen C M M, et al. (2010) Generation of stable monoclonal antibody-producing B cell receptor positive human memory B cells by genetic programming. Nat Med 16: 123-128.

What is claimed is:

1. A transgenic mouse whose genome comprising a nucleic acid construct comprising as least one transgene linked to a promoter effective for expression of human leukocyte antigen DR4, which is capable of developing a functional human immune system upon infusion of human HLA-matched hematopoietic stem cells.

2. The transgenic mouse of claim 1, wherein said genome further a nucleic acid construct comprising a gene linked a promoter effective for expressing human leukocyte antigen A2.1.

3. The transgenic mouse of claim 1, wherein said genome further comprising a nucleic acid construct comprising a gene linked to a promoter effective for expressing of human costimulatory molecules (CD80).

4. The transgenic mouse of claim 1, wherein said genome of said transgenic mouse further comprise one or more knockout mutations for abrogating the mouse immune system.

5. The transgenic mouse of claim 4, wherein said knockout mutation is selected from the group consisting: AbbKO, 1B2m KO, Rag1 KO, and IL2RgcKO.

6. The transgenic mouse of claim 1, wherein said genome of said transgenic mouse further lacks genes encoding for endogenous mouse MHC I and MHC II molecules and mouse T and B cells.

7. A method of generating the transgenic mouse of claim 1, comprising
i) introducing a transgene comprising a nucleotide sequence encoding a human costimulatory molecules CD80 operably linked to a promoter into a mouse fertilized oocyte;
ii) allowing said fertilized oocyte to develop into an embryo;
iii) transferring said embryo into a pseudopregnant female mouse;
iv) allowing said embryo to develop to term;
v) identifying said transgenic mouse whose genome comprising a nucleotide sequence encoding a human costimulatory molecule CD80 and human leukocyte DR4 operably linked to a promoter;
vi) crossbreeding said transgenic mouse of step v with transgenic mouse whose genome comprising nucleotide sequence encoding a human leukocyte A2 operatively linked to a promoter;
vii) intercrossing said transgenic mouse of step vi; and
vii) identifying transgenic mouse whose genome comprising a nucleotide sequence encoding human leukocyte A2 and DR4.
vi) crossbreeding said transgenes with one or more KO mutations that abolish development of mouse immune system consisting the group of AbbKO, Rag KO, IL2RgcKO, and B2m KO.

8. The method of claim 7, wherein genome of said transgenic mouse further lacks genes encoding for endogenous mouse MHC I and MHC II molecules and mouse T and B cells.

9. A method for evaluating an agent for human vaccine use comprising:
i) providing two groups of transgenic mice according to claims 1-3;
ii) administering an agent to one group of said transgenic mouse;
iii) comparing the immunogenic response in said group of transgenic mice with immunogenic response of the group of transgenic mice to which no agent has been administered, wherein an agent that induces a higher immunogenic response is identified as an agent for vaccine use.

10. The method of claim 9, where said immunogenic response is a humoral immune response or a cellular immune response.

11. The method of claim 9, where said agent is selected from the group consisting of: biologics, pharmaceuticals, and chemicals.

12. The method of claim 9, wherein said agent is included in a vaccine further comprising an adjuvant.

13. The method of claim 9, wherein said agent further include a vaccine further comprising a pharmaceutical carrier.

14. A method for producing a fully human antibody specific for a desired antigen, comprising:
i) infusing a transgenic mouse whose genome comprising a nucleic acid construct comprising as least one transgene linked to a promoter effective for expression of human leukocyte antigen DR4, with human HLA-matched hematopoietic stem cells allow it to develop a functional human immune system; and
ii) immunizing said transgenic mouse with a desired antigen;
iii) recovering the antibody.

15. The method of claim 14, wherein the desired antigen is from the group consisting of: leukocyte markers; histocompatibility antigens; integrins; adhesion molecules; interleukins; interleukin receptors; chemokines; growth factors; growth factor receptors; interferon receptors; immunoglobulins and their receptors; tumor antigens; allergens; viral proteins; rickettsial proteins; bacterial proteins/glycoproteins; protozoal proteins/glycoproteins; helminth proteins/glycoproteins; toxins; blood factors; enzymes; ganglioside GD3, ganglioside GM2; LMP1, LMP2; eosinophil major basic protein, eosinophil cationic protein; pANCA; Amadori protein; Type IV collagen; glycated lipids; inter-gamma.-interferon; A7; P-glycoprotein; Fas (AFO-1) and oxidized-LDL.

16. The method of claim 14, wherein said genome of said transgenic mouse further comprising a gene linked to a promoter effective for expressing of HLA-A2.

17. The method of claim 14, wherein said genome of said transgenic mouse further comprising a gene linked to a promoter effective for expressing human costimulatory molecules (CD80).

18. The method of claim 14, wherein said genome of said transgenic mouse further comprising one or more knockout mutations for abrogating the mouse immune system.

19. The method of claim 18, wherein said knockout mutations are selected from the group consisting: AbbKO, 1B2m KO, Rag1KO, and IL2RgcKO.

20. The method of claim 14, wherein said genome of said transgenic mouse further lacks genes encoding for endogenous mouse MHC I and MHC II molecules and mouse T and B cells.

21. Fully human antibody against a specific antigen produced using method of claims 14-20.
